(19) United States
(12) Patent Application Publication
(10) Pub. No.: US 2024/0207559 A1
(43) Pub. Date: Jun. 27, 2024

(54) DEVICE FOR PREVENTING AIRBORNE PATHOGENS FROM ENTERING THE NASAL AIRWAY

(71) Applicant: Andres Mencia, Margate, FL (US)

(72) Inventor: Andres Mencia, Margate, FL (US)

(21) Appl. No.: 18/393,026

(22) Filed: Dec. 21, 2023

Related U.S. Application Data
(60) Provisional application No. 63/434,360, filed on Dec. 21, 2022.

(51) Int. Cl. A61M 16/06; A61M 16/00

(52) U.S. Cl. CPC A61M 16/0672; A61M 16/0003; A61M 16/0683; A61M 2202/0007; A61M 2202/0208; A61M 2205/02; A61M 2205/7509; A61M 2205/7518; A61M 2205/7545; A61M 2210/0618

(57) ABSTRACT

A device for blocking the nasal airway at the opening of the nostrils has two tubes that terminate at distal tips. The tubes are each separated into channels, or lumens, running the length of the tubes, including an outer lumen that extends from an inflation connection port to an inflatable donut just below the distal tip of each tube. With the distal tips received in the respective left and right nostrils of the user, the donuts are inflated to close off the nostrils at the rim, adjacent the nasal vestibule. Filters may be provided between the inflation ports and the distal tips for cleaning air prior to entry into the nasal cavity. The device may further include left and right ear straps to secure the device on the user's ears.

This non-provisional patent application is based on provisional patent application Ser. No. 63/434,360 filed on Dec. 21, 2022.

### BACKGROUND OF THE INVENTION

### Field of the Invention

The present invention relates to a device for closing or blocking the nostrils to prevent entry of airborne pathogens, and more particularly, to a device that includes members that are inserted within the nostrils and expanded to block the nasal airway at the opening of the nostrils.

### Description of the Related Art

For many years, it has been generally accepted that exposure and contraction of colds, flu and other viruses is primarily through hand-to-hand contact or by touching contaminated surfaces and then subsequently introducing the pathogens into the body by contact of one's hands with the mouth, nose and/or eyes. Following the COVID-19 outbreak, it has been learned that the primary means of contracting COVID, as well as the flu virus, is by inhaling tiny airborne droplets of contaminated fluids through the nasal passageways into the nasal cavity, nasal mucosa and lungs. This is due to the fact that many pathogens are most potent when in an airborne state after immediate release from an infected person, whether by normal breathing, coughing or sneezing. Thus, by blocking the nasal cavity, at the opening of the nostrils, entry of harmful pathogens into the nose can be prevented.

### SUMMARY OF THE INVENTION

The present invention is directed to a device for blocking the nasal airway at the opening of the nostrils, adjacent to the nasal vestibule, for preventing entry of harmful pathogens into the nasal cavity, airway and lungs, thereby significantly reducing the likelihood of infection with the flu virus, cold virus (e.g., rhinovirus) and COVID. The device has two tubes that terminate at distal tips. The tubes are each separated into channels, or lumens, running the length of the tubes. A delivery lumen inside of or alongside of each tube extends from an inflation connection port to an inflatable donut. The delivery lumen is separate from a tubular passage inside of the tube. The inflatable donut (balloon) is just below the distal tip of each tube and receives a flow of air or fluid, preferably from a connected syringe, to inflate the donut. The two tubes may share a common inflation port, or they may have separate inflation ports for connecting the syringe or other inflation device. In a preferred embodiment, the donuts are inflated separately with each donut having a separate corresponding inflation port and independent channel or delivery lumen extending from the inflation port to the balloon. This accommodates for asymmetry of the nostrils, wherein one nostril is larger than the other which is very typical. Thus, one donut can be inflated more if the nostril is larger than the other. Moreover, each donut receives a flow of air or liquid on both sides (i.e., opposite sides of the donut) to allow for uniform inflation around the entire donut.

The distal tips are structured and disposed for insertion into the respective left and right nostrils of the user. With the distal tips inserted within the nostrils, the donuts are inflated to close off the nostrils at the rim, adjacent the nasal vestibule. Filters may be provided between the inflation ports and the distal tips for cleaning air prior to entry into the nasal cavity. The device of the present invention may further include left and right ear straps to secure the device on the user's ears.

The structure of the tubes is similar to a Foley catheter. The length of each distal tip is approximately ¼ inch to ½ inch, allowing each tip to be received within the user's nostrils. When inflated, the donuts are large enough to block the nasal entrance, but are sufficiently small in diameter prior to inflation to allow insertion into the nostrils. Once the device is in place within the nostrils, the device may remain in place for an extended period as long as it is not overinflated which could potentially cut off the circulation of the nasal mucosa. When the donuts are inflated to block the opening of the nostrils, airborne pathogens (e.g., COVID, flu virus, cold virus) are prevented from reaching the nasal airway and lungs. Use of the device allows a person to drink beverages and consume food, while being protected without the need for a mask. The device also allows for a person to speak to others without the need to wear a mask over their nose and mouth. For instance, a public figure can hold a conference while wearing the device, with the donuts inflated to protectively close the nostrils, without the need for a mask when the speaker is on a podium in front of a group of people.

### BRIEF DESCRIPTION OF THE DRAWINGS

For a fuller understanding of the nature of the present invention, reference should be had to the following detailed description taken in connection with the accompanying drawings in which:

FIG. 1 is a front perspective view of one embodiment of the present invention shown in relation to a user's nose, prior to insertion of the distal tips within the nostrils and with the donuts deflated;

FIG. 2 is a front perspective view of another embodiment of the present invention;

FIG. 3 is a front perspective view showing yet another embodiment of the present invention, shown in relation to a user's nose, prior to insertion of the distal tips into the user's nostrils;

FIG. 4 is a side elevational view, shown in partial cross-section, and illustrating the distal tip and inflatable donut received within a user's nostril and with the donut inflated to close and seal the nostril at the rim surrounding the entry of the nostril, adjacent the nasal vestibule and thereby preventing airborne pathogens from entering the nose and the nasal cavity;

FIG. 5 is an isolated cross-sectional view showing a delivery lumen extending from the inflation connection port to one of the inflatable donuts for delivering a pressurized flow of air or liquid from a syringe attached to the inflation connection port to the donut in order to inflate the donut when received within the user's nostril; and

FIG. 6 is an isolated cross-sectional view showing a further embodiment of the device of the present invention including both an inflation connection port and a separate oxygen delivery port. Like reference numerals refer to like parts throughout the several views of the drawings.

### DETAILED DESCRIPTION OF THE PREFERRED EMBODIMENTS

Referring to the several views of the drawings, various embodiments of the present invention are shown. The present invention is directed to a device for preventing airborne pathogens from entering the nasal airway through the nostrils. More particularly, the device is structured for blocking the nasal airway at the opening of the nostrils, adjacent to the nasal vestibule, for preventing entry of harmful pathogens into the nasal cavity, airway and lungs, thereby significantly reducing the likelihood of infection with the flu virus, cold virus (e.g., rhinovirus), COVID and other infectious diseases and illnesses.

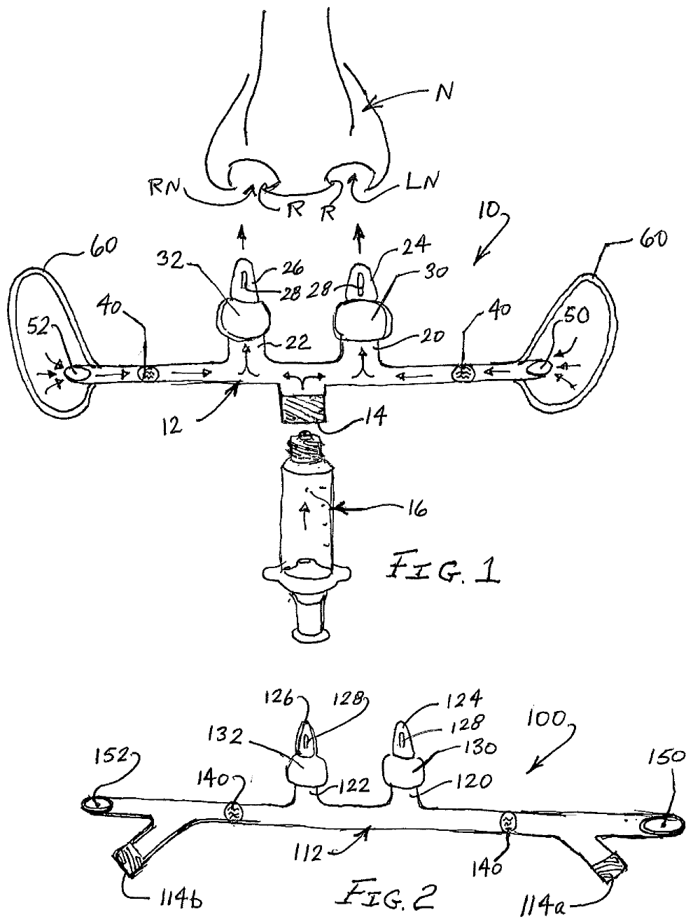

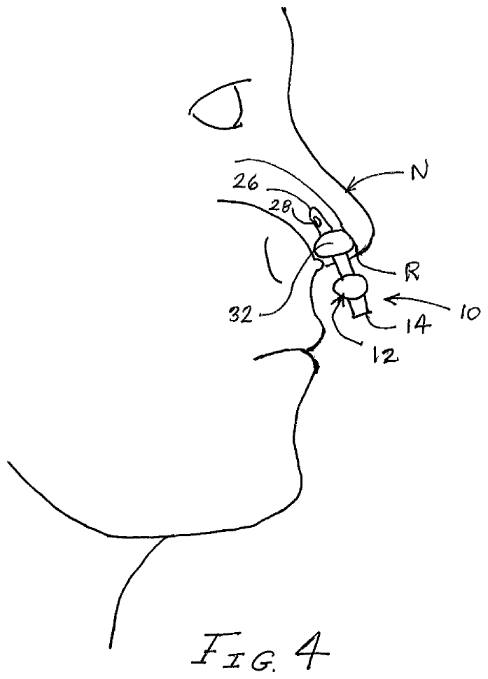

As seen in FIG. 1, a first embodiment of the device of the present invention is shown and is generally indicated as 10. The device 10 includes a tubular manifold 12 having an inflation port 14 at the bottom for attachment of a syringe 16 thereto. A pair of tubes 20, 22 extend upwardly from the manifold 12 and each terminate at a respective distal tip 24, 26. The distal tips of these tubes include an opening 28 for passage of air therethrough. The tubes 20, 22 further include inflatable donuts 30, 32 that are inflated by the syringe 16 attached to the inflation port 14 on the manifold 12. The donuts 30, 32 may be inflated with either air or liquid, such as saline, contained in the syringe. In use, the distal tips 24, 26 are received within the left and right nostrils LN and RN of the user and the donuts 30, 32 are inflated to close off the nostrils, just inside the rim R of the nose N, as shown in FIG. 4. The device 10 according to the embodiment of FIG. 1 further includes air filters 40 for filtering air that enters through opposite open ends 50, 52 of the manifold to be breathed in through the openings 28 of the distal tips 24, 26 received in the nostrils. The filters remove microbes, pathogens, pollutants and other impurities from the air that enters the ends 50, 52. The filters 40 are particularly suited for preventing entry of pathogens and other airborne germs, particles and pollutants into the nose. The device 10 further includes straps 60 for securing the device over the user's ears. The straps may be made of an elastomeric material that can be stretched over the user's ears. The device 10 allows the user to breath freely without allowing airborne pathogens to enter through the user's nostrils.

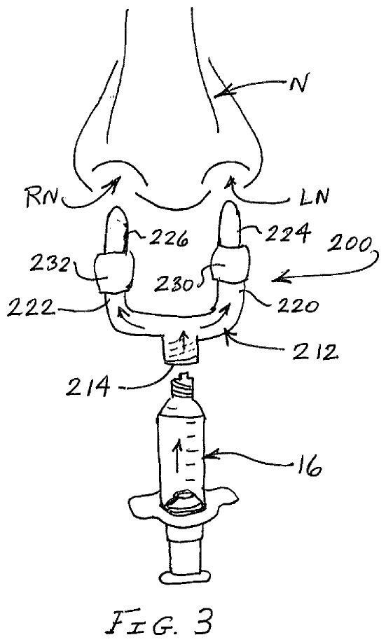

FIG. 2 illustrates another embodiment of the device of the present invention, wherein the device is generally indicated as 100. The device 100 includes a tubular manifold 112 and a pair of spaced tubes 120, 122 with distal tips 124, 126 with openings 128 for receipt within the user's nostrils. Similar to the embodiment of FIG. 1, inflatable donuts 130, 132 on the tubes are inflated with the use of a syringe 16 that attaches to two separate inflation connection ports 114a, 114b. In this embodiment, the inflation ports 114a, 114b may be provided at opposite ends of the tubular manifold 112 to separately inflate the left and right donuts 130, 132 within the users left and right nostrils. Air is able to enter through the opposite ends 150, 152 of the tubular manifold, and is cleaned by the filters 140 prior to entering into the central portion of the manifold and through the tubes 120, 122 into the user's nostrils, through the openings 128 in the distal tips 124, 126.

FIG. 3 is yet a further embodiment of the present invention which shows a simplified version of the device, generally indicated as 200. The device 200 including a central tubular manifold 212, with an inflation connection port 214, and spaced tubes 220, 222 having distal tips 224, 226 for receipt within the user's nostrils. Similar to the embodiments of FIGS. 1 and 2, inflatable donuts 230, 232 are provided on the spaced tubes 220, 222 and are inflated by the syringe 16, injecting either air or a liquid through the inflation port 214, through the manifold 212 and to the donuts 230, 232.

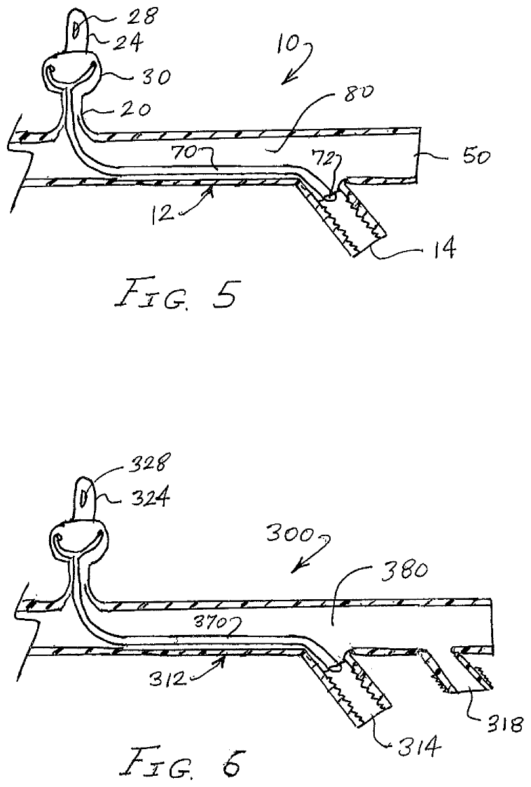

Referring to FIG. 5, a cross sectional view illustrates the delivery of air or liquid from the inflation connection port 14 to one of the inflatable donuts 30, 32 in any of the embodiments described above. In particular, a delivery lumen 70 extends from the inflation connection port to a respective one of the inflatable donuts. The lumen 70 splits into two branches at the donut in order to release air or liquid on opposite sides of the donut for uniform inflation. An opposite end of the lumen 70 is fitted to a valve 72 within the inflation connection port. When the syringe 16 is threadably attached within the inflation connection port, a tip of the syringe engages the valve 72, causing the valve 72 to open so that air or liquid within the syringe can be delivered into the lumen 70 for delivery to the inflatable donut. In order to deflate the donut, the syringe can be attached to the inflation connection port, thereby opening the valve 72, whereupon air or liquid can be withdrawn from the donut, through the lumen 70 and into the syringe. The user may also breathe through the nostrils so that ambient air is drawn through one of the openings at the end of the tubular manifold 12, such as at 50, and through the opening 28 on the distal tip 24. The air that is drawn through the air inlet opening 50 travels through an air passage lumen 80 that is in airflow communication between the air inlet opening 50 and the opening 28 on the distal tip 24. Thus, air that is drawn through the air inlet opening 50 travels through the air passage lumen 80 and to the opening 28 on the distal tip 24 for inhalation within the user's nostril. As described above, a filter may be provided within the air passage lumen 80 or at the air inlet opening 50, as well as the air inlet opening 52 on the opposite side of the tubular manifold.

Referring to FIG. 6, a further embodiment of the device of the present invention is shown and is generally indicated as 300. The device 300 includes a central tubular manifold 312 with an inflation connection port 314 and a separate oxygen delivery connection port 318. The inflation connection port 314 is connected in fluid communication with delivery lumen 370 and operates in the same manner as described above in connection with the previous embodiments to inflate and deflate the respective inflatable donut for the left and right nostrils. The oxygen delivery connection port 318 allows for connection of an oxygen supply, such as from a pressurized oxygen canister or an oxygen generating machine. Oxygen delivered from the oxygen supply source travels through the oxygen delivery connection port 318, and through the second lumen 380 within the tubular manifold 312 for delivery to the respective one of the distal tips, such as 324 for inhalation of the oxygen by the user through the nose. More particularly, the oxygen exits the opening 328 in the distal tip 324 and into the user's nasal passageway, whereupon breathing in through the nose allows the user to inhale the oxygen from the oxygen supply source.

In each of the embodiments described above and illustrated in FIGS. 1-6, the tubular manifold is flexible so that it can bend and wrap around the sides of the user's face, below the nose and towards the opposite ears. In one embodiment, the tubular manifold is formed of a plastic material that is flexible and resilient. The device, and particularly the tubular manifold, the pair of tubes, the inflatable donuts and the distal tips can be made in various colors to be more appealing. Also, in each of the above described embodiments, the delivery lumens may be formed of a plastic material that is flexible, but of a different consistency and stronger than the tubular manifold. The inflatable donuts are formed or an elastomeric material, such as a rubber composition, to allow for expansion and retraction upon inflation and deflation.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A device for blocking the nasal airway at the opening of the nostrils, the device comprising:
a main tubular manifold having a plurality of lumens extending therethrough for allowing passage of air or liquid therethrough, and the plurality of lumens including at least one delivery lumen, and the main tubular manifold including at least one inflation port communicating with the at least one delivery lumen, and the inflation port being structured and disposed for threaded attachment of a syringe thereto for injection and withdrawal of air or liquid within the at least one delivery lumen;
a pair of tubes extending upwardly from the manifold and each terminating at a respective distal tip, and the distal tip of each of the pair of tubes being structured and disposed for insertion within a respective left or right nostril of the user;
an inflatable donut on each of the pair of tubes and structured and disposed for inflation and expansion to block the nasal airway at the opening of the respective nostril; and
the delivery lumen extending from the at least one inflation port to at least one of the inflatable donuts for delivering a pressurized charge of air or liquid into the at least one inflatable donut for inflation and expansion thereof, and the delivery lumen further being structured and disposed for directing withdrawal of the charge of air or liquid from the at least one inflatable donut and out through the inflation port for deflating and retracting the donut.

2. The device as recited in claim 1 further comprising:
at least one opening in each of the respective distal tips of the pair of tubes;
the plurality of lumens further including at least one air passage lumen communicating with the at least one opening in each of the respective distal tips; and
at least one air entry opening in the main tubular manifold and communicating with the air passage lumen for allowing the user to inhale through the nostrils wherein ambient air enters through the at least one air opening in the main tubular manifold and flows through the air passage lumen and the at least one opening in the distal tips when the user breathes through the nostrils.

3. The device as recited in claim 2 further comprising:
at least one filter between the at least one air opening in the main tubular manifold and the at least one opening in each of the respective distal tips for filtering microbes, pathogens and other impurities from the ambient air when the user inhales through the nostrils.

4. The device as recited in claim 3 further comprising:
first and second ear straps attached to the main tubular manifold for securing the device to the user with the first and second straps fitted on the user's left and right ears.

5. The device as recited in claim 4 wherein the first and second straps are formed of an elastomeric material.

6. The device as recited in claim 5 further comprising:
at least one oxygen supply connection port on the main tubular manifold and communicating with the air passage lumen, and the at least one oxygen supply connection port being structured and disposed for releasable connection of an oxygen supply source thereto for delivering oxygen to the user through the nostrils.

7. The device as recited in claim 6 wherein the delivery lumen splits into two branches at the respective inflatable donut in order to release air or liquid on opposite sides of the inflatable donut for uniform inflation thereof.

8. The device as recited in claim 7 further comprising:
a first inflation port communicating with a first delivery lumen and a first inflatable donut on one of the pair of tubes;
a second inflation port communicating with a second delivery lumen and a second inflatable donut on the other of the pair of tubes; and
wherein the first inflatable donut and the second inflatable donut are inflated separately and independently via the respective first and second inflation ports.

9. The device as recited in claim 2 further comprising:
at least one filter at the at least one air opening in the main tubular manifold for filtering microbes, pathogens and other impurities from the ambient air when the user inhales through the nostrils.

10. A device for blocking the nasal airway at the opening of the nostrils, the device comprising:
a main tubular manifold having a plurality of lumens extending therethrough for allowing passage of air or liquid therethrough, and the plurality of lumens including at least one delivery lumen, and the main tubular manifold including at least one inflation port communicating with the at least one delivery lumen, and the inflation port being structured for connection to a pressurized airflow source for injection and withdrawal of air within the at least one delivery lumen;
a pair of tubes extending upwardly from the manifold and each terminating at a respective distal tip, and the distal tip of each of the pair of tubes being structured and disposed for insertion within a respective left or right nostril of the user;
an inflatable donut on each of the pair of tubes and structured and disposed for inflation and expansion to block the nasal airway at the opening of the respective nostril; and
the delivery lumen extending from the at least one inflation port to at least one of the inflatable donuts for delivering a pressurized charge of air from the pressurized airflow source into the at least one inflatable donut for inflation and expansion thereof, and the delivery lumen further being structured and disposed for directing withdrawal of the charge of air or liquid from the at least one inflatable donut and out through the inflation port for deflating and retracting the donut.

11. The device as recited in claim 10 further comprising:
at least one opening in each of the respective distal tips of the pair of tubes;
the plurality of lumens further including at least one air passage lumen communicating with the at least one opening in each of the respective distal tips; and
at least one air entry opening in the main tubular manifold and communicating with the air passage lumen for allowing the user to inhale through the nostrils wherein ambient air enters through the at least one air opening in the main tubular manifold and flows through the air passage lumen and the at least one opening in the distal tips when the user breathes through the nostrils.

12. The device as recited in claim 11 further comprising:
at least one filter between the at least one air opening in the main tubular manifold and the at least one opening in each of the respective distal tips for filtering microbes, pathogens and other impurities from the ambient air when the user inhales through the nostrils.

13. The device as recited in claim 12 further comprising:
first and second ear straps attached to the main tubular manifold for securing the device to the user with the first and second straps fitted on the user's left and right ears.

14. The device as recited in claim 13 wherein the first and second straps are formed of an elastomeric material.

15. The device as recited in claim 14 further comprising:
at least one oxygen supply connection port on the main tubular manifold and communicating with the air passage lumen, and the at least one oxygen supply connection port being structured and disposed for releasable connection of an oxygen supply source thereto for delivering oxygen to the user through the nostrils.
